(19) United States
(12) Patent Application Publication
(10) Pub. No.: US 2026/0326060 A1
(43) Pub. Date: Oct. 22, 2026

(54) REACTOR

(71) Applicant: HONDA MOTOR CO., LTD., Tokyo (JP)

(72) Inventor: Hideaki SUMI, Saitama-ken (JP)

(21) Appl. No.: 19/491,036

(22) PCT Filed: Mar. 27, 2024
(86) PCT No.: PCT/JP2024/012456

§ 371 Date: Dec. 8, 2025

(30) Foreign Application Priority Data
Jun. 14, 2023 (JP) 2023-097620

(51) Int. Cl. B01J 8/06

(52) U.S. Cl. CPC B01J 8/067; B01J 8/065; B01J 2208/00115; B01J 2208/0053

(57) ABSTRACT

A reactor 1 for producing a predetermined product while a predetermined source gas is introduced and an exothermic reaction occurs due to a catalytic action of a predetermined catalyst 5 includes a casing 2 including a source gas introduction port 12a into which the source gas is introduced and a product taking-out port 13b for taking out the product produced, the casing 2 being filled with the catalyst 5, a control refrigerant pipe 3 disposed in the casing 2 so that a control refrigerant flows in the control refrigerant pipe 3 to control a reaction temperature in the casing 2, a condensing refrigerant pipe 4 disposed in the casing 2 so that a condensing refrigerant flows in the condensing refrigerant pipe 4 to cause condensation of the product on an outer peripheral surface of a longitudinal pipe portion 4a, and a product guiding path 13c provided in the casing 2 and guiding the product condensed and attached to the outer peripheral surface of the longitudinal pipe portion 4a to the product taking-out port 13b.

### CROSS-REFERENCE TO RELATED APPLICATION(S)

This is a national stage application filed under 37 U.S.C. 371 based on International Patent Application No. PCT/JP2024/012456, filed Mar. 27, 2024, which claims priority to Japanese Patent Application No. 2023-097620 filed Jun. 14, 2023, the disclosures of which are incorporated herein by reference in their entireties.

### TECHNICAL FIELD

The present invention relates to a reactor for producing a predetermined product while a predetermined source gas is introduced and an exothermic reaction occurs due to a catalytic action of a predetermined catalyst.

BACKGROUND ART

In recent years, in order to reduce adverse effects on the global environment, vehicle emissions control has further progressed. In particular, carbon dioxide contained in emissions of an internal combustion engine is said to be a cause of global warming, and reduction of carbon dioxide emission is required.

In order to effectively utilize the carbon dioxide as described above, Patent Literature 1 discloses a reaction device in which a source gas containing hydrogen and carbon dioxide is introduced into a reactor filled with a predetermined catalyst to produce hydrocarbon in the reactor.

In this reaction device, a first catalyst unit that generates carbon monoxide from the source gas is disposed on the upstream side of the reactor, and a second catalyst unit that generates hydrocarbon using the generated carbon monoxide and hydrogen is disposed on the downstream side of the reactor. In the above-described reactor, while a reverse shift reaction occurs in the first catalyst unit on the upstream side, a Fischer-Tropsch (FT) reaction occurs in the second catalyst unit on the downstream side. This FT reaction is an exothermic reaction, and in particular, since the exotherm concentrates on the upstream portion of the second catalyst unit, the temperature non-uniformity in the second catalyst unit is significant. In order to reduce such temperature non-uniformity, an inert catalyst is mixed in the upstream portion of the second catalyst unit, whereby, in the above-described reaction device, the reaction in the second catalyst unit is stabilized by reducing the temperature difference in the entire second catalyst unit.

### CITATION LIST

### Patent Literature

Patent Literature 1: JP 2022-102704 A

### SUMMARY OF INVENTION

### Technical Problem

In the reactor, when the reaction proceeds and the concentration of the product increases, the degree of progress of the reaction decreases. In particular, when the reaction is in an equilibrium state, the reaction itself does not proceed. As a result, in the reactor, the recovery efficiency of the product and the reaction rate may decrease.

The present invention has been made to solve the above-described problems, and an object thereof is to provide a reactor capable of efficiently recovering a product and improving a reaction rate.

### Solution to Problem

In order to achieve the above object, an invention according to claim 1 is a reactor 1 for producing a predetermined product while a predetermined source gas is introduced and an exothermic reaction occurs due to a catalytic action of a predetermined catalyst 5, and includes a casing 2 including a source gas introduction port 12a into which the source gas is introduced and a product taking-out port 13b for taking out the product produced, the casing 2 being filled with the catalyst, a first refrigerant flow path (control refrigerant pipe 3 in the present embodiment (the same applies in this paragraph)) disposed in the casing so that a predetermined first refrigerant (control refrigerant) flows in the first refrigerant flow path to control a reaction temperature in the casing, a second refrigerant flow path (condensing refrigerant pipe 4) disposed in the casing so that a predetermined second refrigerant (condensing refrigerant) flows in the second refrigerant flow path to cause condensation of the product on an outer peripheral surface of the second refrigerant flow path, and a product guiding path 13c provided in the casing and guiding the product condensed and attached to the outer peripheral surface of the second refrigerant flow path to the product taking-out port.

According to this configuration, the casing of the reactor is filled with the predetermined catalyst, and the casing is provided with the first refrigerant flow path for controlling the reaction temperature, the second refrigerant flow path for causing condensation of the product on the outer peripheral surface, and the product guiding path for guiding the condensed product to the product taking-out port. When the predetermined source gas is introduced into the casing through the source gas introduction port, the exothermic reaction occurs due to the catalytic action of the catalyst, and the predetermined product is generated while the reaction temperature is controlled by the first refrigerant flowing through the first refrigerant flow path. In addition, in this case, the second refrigerant flows through the second refrigerant flow path to cause condensation of the product on the outer peripheral surface of the second refrigerant flow path.

That is, the produced gaseous product is condensed, and the liquid product is attached to the outer peripheral surface of the second refrigerant flow path. The condensed product is guided to the product taking-out port of the casing via the product guiding path, and is taken out to the outside.

As described above, the gaseous product generated by the reaction in the casing of the reactor is condensed and attached to the outer peripheral surface of the second refrigerant flow path to change the state to a liquid state, whereby the product can be recovered efficiently. In addition, since the concentration of the gaseous product in the casing can be reduced, the degree of progress of the reaction performed in the casing can be maintained at a high level, thereby improving the reaction rate more than that in the conventional reactor.

An invention according to claim 2 is the reactor according to claim 1, wherein a temperature of the second refrigerant is set to be lower than a temperature of the first refrigerant.

According to this configuration, the reaction is promoted while the reaction temperature in the casing is controlled by the first refrigerant, and the gaseous product is condensed by the second refrigerant having a temperature lower than that of the first refrigerant to cause condensation of the gaseous product on the outer peripheral surface of the second refrigerant flow path. In this manner, according to the above-described configuration, it is possible to achieve both the promotion of the reaction in the casing and the condensation of the product.

An invention according to claim 3 is the reactor according to claim 1, wherein the casing includes a main body portion 11 formed to have a tubular shape extending in an up-down direction, an upper wall portion 12 which closes an upper end portion of the main body portion and is provided with the source gas introduction port, and a lower wall portion 13 which closes a lower end portion of the main body portion and is provided with the product taking-out port, wherein the second refrigerant flow path includes a plurality of longitudinal flow path portions (longitudinal pipe portions 4a) extending in the up-down direction in the main body portion and through which the second refrigerant flows, wherein the first refrigerant flow path includes a plurality of catalyst holding portions 3c each formed to surround each of the plurality of longitudinal flow path portions in a state of being spaced from an outer peripheral surface of each of the longitudinal flow path portions by a predetermined distance, and is configured such that the first refrigerant flows to an outer peripheral portion of each of the catalyst holding portions, and wherein the catalyst is filled between each of the longitudinal flow path portions and each of the catalyst holding portions.

According to this configuration, the casing includes the main body portion formed to have a tubular shape extending in the up-down direction, and the upper end portion and the lower end portion of the main body portion are closed by the upper wall portion and the lower wall portion, respectively. In addition, the second refrigerant flow path includes the plurality of longitudinal flow path portions extending in the up-down direction in the main body portion of the casing and through which the second refrigerant flows. Further, the first refrigerant flow path includes the plurality of catalyst holding portions each formed to surround each of the plurality of longitudinal flow path portions of the second refrigerant flow path and is configured such that the first refrigerant flows to the outer peripheral portion of each of the catalyst holding portions. The catalyst is filled between each of the longitudinal flow path portions and each of the catalyst holding portions. Since the first refrigerant flows to the outer peripheral portion of each of the catalyst holding portions of the first refrigerant flow path, the reaction temperature of the source gas passing through the catalyst filled between the longitudinal flow path portion and the catalyst holding portion can be controlled appropriately. In addition, since the second refrigerant flows through each of the longitudinal flow path portions of the second refrigerant flow path, the gaseous product generated in the catalyst holding portion can be easily condensed on the outer peripheral surface of each of the longitudinal flow path portions.

An invention according to claim 4 is the reactor according to claim 3, wherein each of the catalyst holding portions is provided with a longitudinal flow path portion cover (longitudinal pipe portion cover 6) that has a tubular shape separating the catalyst in the catalyst holding portion from the longitudinal flow path portion in a state of extending along the longitudinal flow path portion and having a predetermined gap from the outer peripheral surface of the longitudinal flow path portion, and that allows passage of gas and prevents contact of the catalyst with the longitudinal flow path portion.

According to this configuration, each of the catalyst holding portions is provided with the longitudinal flow path portion cover that has a tubular shape separating the catalyst from the longitudinal flow path portion in the state of extending along the longitudinal flow path portion and having the predetermined gap from the outer peripheral surface of the longitudinal flow path portion. The longitudinal flow path portion cover is configured to allow passage of gas and to prevent contact of the catalyst in the catalyst holding portion with the longitudinal flow path portion. Accordingly, the liquid product condensed and attached to the outer peripheral surface of the longitudinal flow path portion can fall along the outer peripheral surface s of the longitudinal flow path portion without leaking to the side provided with the catalyst, and can be recovered efficiently.

An invention according to claim 5 is the reactor according to claim 4, wherein the longitudinal flow path portion cover is made of a punching plate in which a large number of through holes having a predetermined diameter are formed.

According to this configuration, by forming each through hole of the punching plate so as to have a diameter that does not allow passage of the catalyst, it is possible to easily obtain the longitudinal flow path portion cover that allows the gaseous product generated in the catalyst holding portion to move from the catalyst side to the longitudinal flow path portion side.

An invention according to claim 6 is the reactor according to any one of claims 3 to 5, wherein the casing further includes a post-reaction gas discharge port 13a for discharging a post-reaction gas in the lower wall portion, and wherein the product taking-out port is provided at a predetermined position shifted in a radial direction in the lower wall portion of the casing and different from that of the post-reaction gas discharge port.

According to this configuration, since the post-reaction gas discharge port is provided in the lower wall portion of the casing, the post-reaction gas remaining in the casing is smoothly discharged to the outside through the post-reaction gas discharge port. In addition, since the product taking-out port is provided at the predetermined position shifted in the radial direction in the lower wall portion of the casing and different from that of the post-reaction gas discharge port, the liquid product is smoothly taken out to the outside through the product taking-out port without being mixed with the post-reaction gas.

### BRIEF DESCRIPTION OF DRAWINGS

FIG. 1(a)-1(e) illustrate sectional views of a reactor according to an embodiment of the present invention. FIG. 1(a) is a longitudinal sectional view of the reactor, and FIG. 1(b), 1(c), 1(d), and 1(e) are transverse sectional views of the reactor illustrated in FIG. 1(a) taken along lines b-b, c-c, d-d, and e-e, respectively.

FIG. 2(a)-2(c) illustrate a reaction operation in the reactor. FIG. 2(a) is a longitudinal sectional view of the reactor, and FIG. 2(b) and 2(c) are transverse sectional views of the reactor illustrated in FIG. 2(a) taken along lines b-b and c-c, respectively.

FIG. 3(a)-3(e) illustrate a flow of gas and condensation of a product in a catalyst holding portion in the reactor. FIG. 3(a) is a longitudinal sectional view of the reactor, FIG. 3(b) is an enlarged view of a longitudinal pipe section and its periphery surrounded by the one-dot chain line in FIG. 3(a), and FIG. 3(c), 3(d), and 3(e) are transverse sectional views of the enlarged view illustrated in FIG. 3(b) taken along lines c-c, d-d, and e-e, respectively.

### DESCRIPTION OF EMBODIMENTS

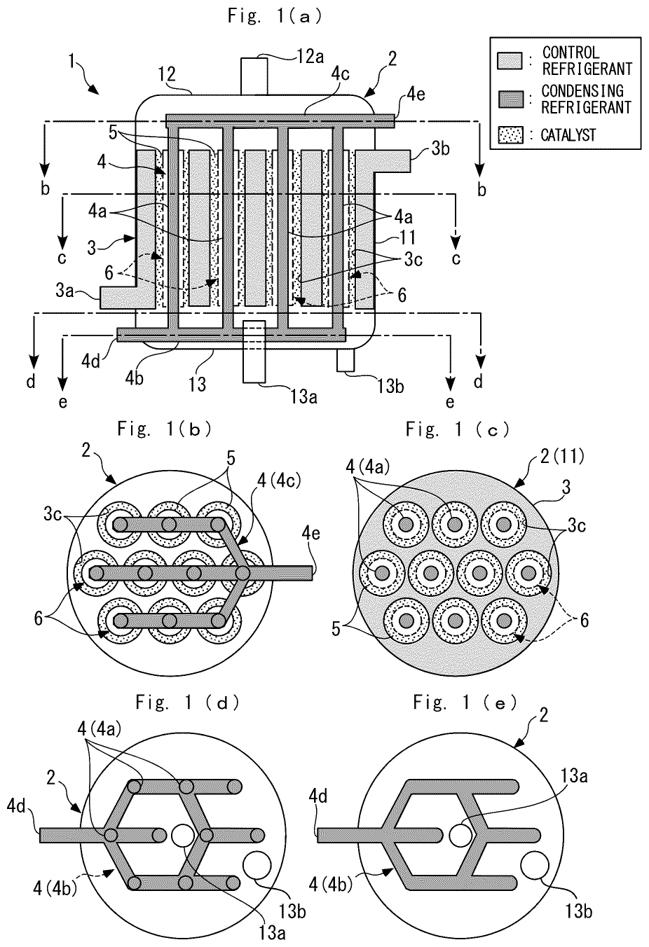

Hereinafter, a preferred embodiment of the present invention will be described in detail with reference to the drawings. FIG. 1(a) is a longitudinal sectional view of a reactor according to an embodiment of the present invention, and FIG. 1(b), 1(c), 1(d), and 1(e) are transverse sectional views of the reactor illustrated in FIG. 1(a) taken along lines b-b, c-c, d-d, and e-e, respectively.

A reactor 1 is configured, for example, to produce a predetermined product (for example, a useful compound such as hydrocarbon or alcohol) while a predetermined source gas (for example, a mixed gas of H₂ (hydrogen) and CO (carbon monoxide) or CO₂ (carbon dioxide)) is introduced and an exothermic reaction occurs inside.

As illustrated in FIG. 1, the reactor 1 includes a casing 2 extending in the up-down direction, a control refrigerant pipe 3 (first refrigerant flow path) through which a refrigerant (first refrigerant) (hereinafter, referred to as “control refrigerant”) for controlling a reaction temperature in the casing 2 flows, a condensing refrigerant pipe 4 (second refrigerant flow path) through which a refrigerant (second refrigerant) (hereinafter, referred to as “condensing refrigerant”) for condensing a product generated by a reaction flows, and a pellet-like catalyst 5 filled in the casing 2.

The casing 2 includes a main body portion 11 formed to have a tubular shape (cylindrical shape in the present embodiment) extending in the up-down direction by a predetermined length, an upper wall portion 12 which closes an upper end portion of the main body portion 11 and is provided with a source gas introduction port 12a, and a lower wall portion 13 which closes a lower end portion of the main body portion 11 and is provided with a post-reaction gas discharge port 13a and a product taking-out port 13b.

The source gas introduction port 12a and the post-reaction gas discharge port 13a are provided at the center portions of the upper wall portion 12 and the lower wall portion 13, respectively. In addition, the product taking-out port 13b is provided at a predetermined position shifted in the radial direction from the center of the lower wall portion 13 and different from that of the post-reaction gas discharge port 13a. In order to prevent a condensed liquid product from being discharged from the post-reaction gas discharge port 13a, the post-reaction gas discharge port 13a is configured such that an upper end portion thereof protrudes to a position higher than a lower lateral pipe portion 4b, to be described later, of the condensing refrigerant pipe 4 and opens upward.

The control refrigerant pipe 3 is provided in the casing 2, and an inflow port 3a for the control refrigerant is provided in the lower portion of the casing 2 while an outflow port 3b is provided in the upper portion of the casing 2. Note that it is also possible to change the positional relationship in the up-down direction between the inflow port 3a and the outflow port 3b depending on the method and conditions of using the reactor 1, the reaction conditions, and the like. In addition, the control refrigerant pipe 3 is formed to surround a plurality of (ten in the present embodiment) catalyst holding portions 3c each of which is a through hole extending by a predetermined length in the up-down direction and having a certain diameter.

The condensing refrigerant pipe 4 includes a plurality of (ten in the present embodiment) longitudinal pipe portions 4a (longitudinal flow path portions) that penetrate the above-described catalyst holding portions 3c of the control refrigerant pipe 3 and are arranged to extend in the up-down direction, a lower lateral pipe portion 4b that connects lower end portions of the respective longitudinal pipe portions 4a with each other and extends horizontally, and an upper lateral pipe portion 4c that connects upper end portions of the respective longitudinal pipe portions 4a with each other and extends horizontally. The lower lateral pipe portion 4b is provided with an inflow port 4d for the condensing refrigerant while the upper lateral pipe portion 4c is provided with an outflow port 4e for the condensing refrigerant. Note that it is also possible to change the positional relationship in the up-down direction between the inflow port 4d and the outflow port 4e depending on the method and conditions of using the reactor 1, the reaction conditions, and the like.

Each of the longitudinal pipe portions 4a of the condensing refrigerant pipe 4 is provided with a longitudinal pipe portion cover 6 extending in the up-down direction so as to cover the outer periphery thereof. The longitudinal pipe portion cover 6 is made of a punching plate in which a large number of through holes having a predetermined diameter are formed, and is formed to have a cylindrical shape having an inner diameter slightly larger than the outer diameter of the longitudinal pipe portion 4a and an outer diameter smaller than the diameter of the catalyst holding portion 3c of the control refrigerant pipe 3. Each of the through holes of the longitudinal pipe portion cover 6 is formed to have a size that allows passage of gas and does not allow passage of pellets of the catalyst 5.

As the catalyst 5, depending on the source gas and the product, a material that promotes a reaction when the product is produced (for example, Fe (iron), Zr (zirconium), Ga (gallium), and/or Na (sodium)) is adopted. As described above, each of the catalysts 5 is formed into a pellet shape having a predetermined size. The catalyst 5 is filled in each catalyst holding portion 3c of the control refrigerant pipe 3 in the casing 2 of the reactor 1. Specifically, in each catalyst holding portion 3c, the catalyst 5 is filled between the inner peripheral surface of the catalyst holding portion 3c and the longitudinal pipe portion cover 6 surrounding the longitudinal pipe portion 4a of the condensing refrigerant pipe 4. As illustrated in FIG. 1(a), on the upper side and the lower side of the control refrigerant pipe 3 in the casing 2, hollow spaces not filled with the catalyst 5 are provided. Accordingly, the reaction temperature in each catalyst holding portion 3c can favorably be controlled by the control refrigerant flowing in the control refrigerant pipe 3, and the source gas introduced from the source gas introduction port 12a can favorably flow to each catalyst holding portion 3c.

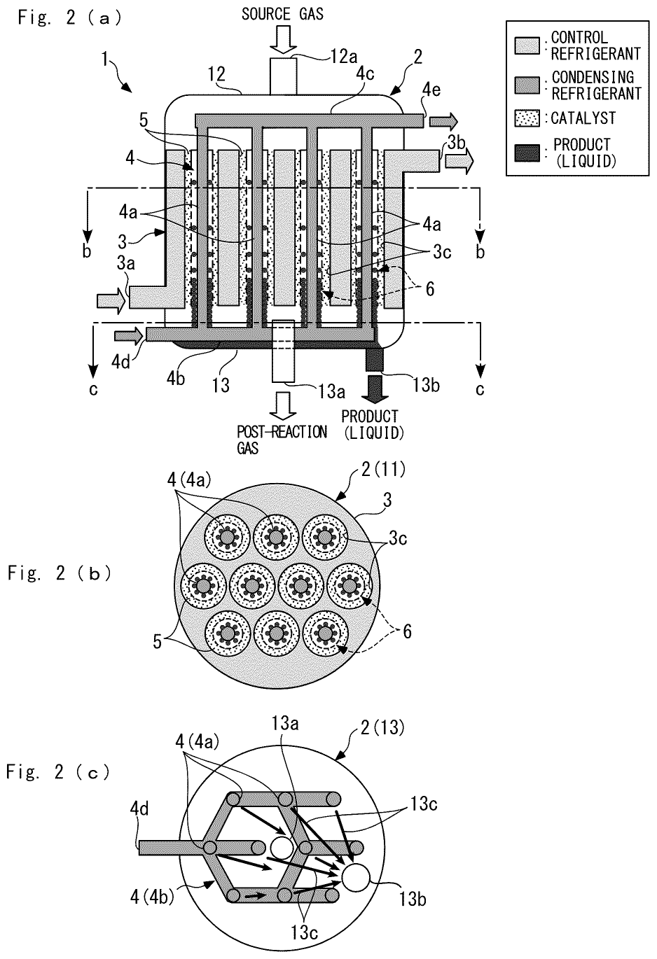

Next, one reaction example in the reactor 1 configured as described above will be described with reference to FIG. 2. In the present reaction example, a source gas which is a mixed gas of H₂ and CO₂ is introduced into the reactor 1, and a direct FT reaction, which is an exothermic reaction, is generated to produce hydrocarbon.

As illustrated in FIG. 2(a), the source gas is introduced into the reactor 1 through the source gas introduction port 12a at the upper portion. In this case, the inside of the reactor 1 is pressurized, and the control refrigerant having a predetermined temperature (for example, 200 to 300 °C) flows into the control refrigerant pipe 3 from the lower inflow port 3a toward the upper outflow port 3b, that is, from bottom to top of the reactor 1. As a result, in the reactor 1, the direct FT reaction occurs at a predetermined pressure (for example, 3 MPa) and a predetermined temperature (for example, 380 °C), and predetermined hydrocarbon (for example, octane) and water are generated in a gaseous state. The control refrigerant flowing through the control refrigerant pipe 3 flows out of the outflow port 3b, is then cooled by a not-illustrated cooling device, and circulates so as to flow into the inflow port 3a again.

In addition, in this case, the condensing refrigerant having a predetermined temperature (200 °C or lower) lower than that of the control refrigerant flows through the condensing refrigerant pipe 4 from the lower inflow port 4d toward the upper outflow port 4e. Specifically, the condensing refrigerant flowing into the inflow port 4d passes through the lower lateral pipe portion 4b, the ten longitudinal pipe portions 4a, and the upper lateral pipe portion 4c of the condensing refrigerant pipe 4 in this order, and flows out of the outflow port 4e. The condensing refrigerant flowing through the condensing refrigerant pipe 4 flows out of the outflow port 4e, is then cooled by a not-illustrated cooling device, and circulates so as to flow into the inflow port 4d again.

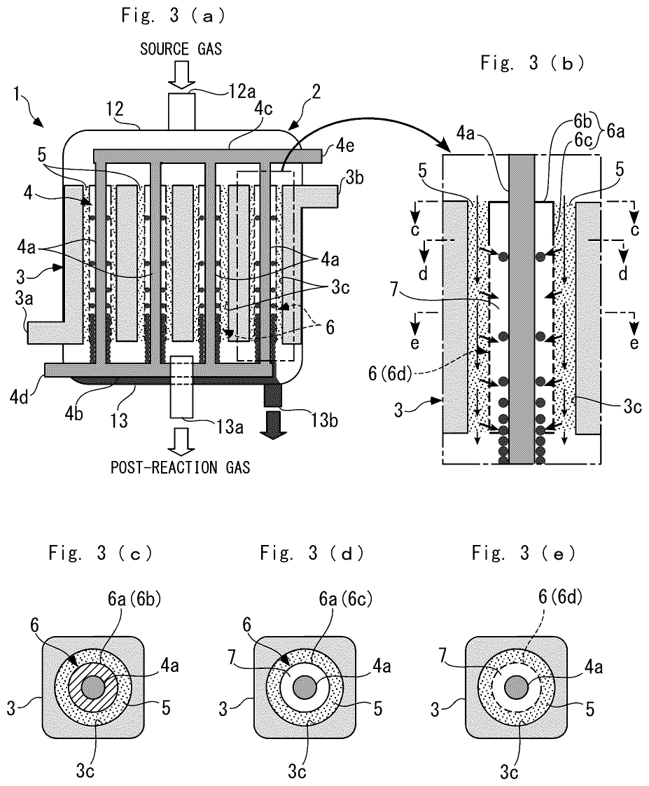

FIG. 3(a) and 3(b) illustrate the flows of the gas and the product and the state of condensation of the product when the direct FT reaction occurs in the catalyst holding portion 3c in the reactor 1. As illustrated in FIG. 3(b), in the longitudinal pipe portion cover 6 provided so as to cover the outer periphery of each longitudinal pipe portion 4a, an upper end portion 6a thereof is not provided with the through holes formed through the punching plate. That is, the upper end portion 6a of the longitudinal pipe portion cover 6 has a top plate portion 6b through which the corresponding longitudinal pipe portion 4a penetrates, and an upper end cylindrical portion 6c which is connected to a peripheral edge portion of the top plate portion 6b and extends downward by a predetermined length. The longitudinal pipe portion cover 6 has a cylindrical cover main body portion 6d that is connected to a lower end peripheral edge portion of the upper end cylindrical portion 6c and extends relatively long to the vicinity of the lower end of the control refrigerant pipe 3, and the cover main body portion 6d is provided with a large number of through holes formed through the punching plate. A condensation space 7 having an annular transverse section is defined between the upper end cylindrical portion 6c and the cover main body portion 6d of the longitudinal pipe portion cover 6 described above and the longitudinal pipe portion 4a located inside the upper end cylindrical portion 6c and the cover main body portion 6d.

As described above, when the source gas is introduced into the reactor 1, the source gas flows downward while being in contact with the catalyst 5 filled in the catalyst holding portion 3c as indicated by the downward arrow in FIG. 3(b). In this case, the gaseous hydrocarbon and water generated in the catalyst holding portion 3c pass through the large number of through holes in the cover main body portion 6d of the longitudinal pipe portion cover 6, pass through the condensation space 7, and move toward the longitudinal pipe portion 4a as indicated by the inclined arrow heading for the longitudinal pipe portion 4a in FIG. 3(b).

Since the upper end portion 6a of the longitudinal pipe portion cover 6 is not provided with the through holes formed through the punching plate, it is possible to prevent the source gas flowing into the catalyst holding portion 3c from above from directly flowing into the condensation space 7 or flowing into the condensation space 7 with almost no contact with the catalyst 5.

When the condensing refrigerant flows through the condensing refrigerant pipe 4, the gaseous hydrocarbon and water that have moved toward the longitudinal pipe portion 4a are condensed and attached to the outer peripheral surface of the longitudinal pipe portion 4a. In the following description, when the condensed hydrocarbon and water are not distinguished, they are collectively referred to as a product.

The liquid product attached to the outer peripheral surface of each of the longitudinal pipe portions 4a falls down along the outer peripheral surface of the longitudinal pipe portion 4a by its own weight. Then, in FIG. 2(c), as indicated by the arrow of a product guiding path 13c on the lower wall portion 13, the liquid product is guided to the product taking-out port 13b and taken out to the outside. The product guiding path 13c is not tubular but is formed such that the upper surface of the lower wall portion 13 is inclined frontward and downward toward the product taking-out port 13b. Therefore, the liquid product fallen on the upper surface of the lower wall portion 13 automatically flows toward the product taking-out port 13b and is taken out through the product taking-out port 13b.

Note that the post-reaction gas remaining in the casing 2 is discharged to the outside through the post-reaction gas discharge port 13a provided in the lower wall portion 13 of the casing 2.

As described above in detail, according to the reactor 1 of the present embodiment, when the source gas is introduced into the casing 2 through the source gas introduction port 12a, the exothermic reaction of the direct FT reaction occurs due to the catalytic action of the catalyst 5 and the reaction temperature control of the control refrigerant, and the gaseous product (hydrocarbon and water) is generated. In this case, when the condensing refrigerant flows through the condensing refrigerant pipe 4, the produced gaseous product condenses, and the liquid product is attached to the outer peripheral surface of the longitudinal pipe portion 4a of the condensing refrigerant pipe 4. The condensed product is guided to the product taking-out port 13b via the product guiding path 13c, and is taken out to the outside.

As described above, the gaseous product generated by the direct FT reaction in the casing 2 of the reactor 1 is condensed and attached to the outer peripheral surface of the longitudinal pipe portion 4a of the condensing refrigerant pipe 4 to change the state to a liquid state, whereby the product can be recovered efficiently. In addition, since the concentration of the gaseous product in the casing 2 can be reduced, the degree of progress of the direct FT reaction performed in the casing 2 can be maintained at a high level, thereby improving the reaction rate more than that in the conventional reactor.

In the reactor 1, the direct FT reaction is promoted while the reaction temperature in the casing 2 is controlled by the control refrigerant, and the gaseous product is condensed by the condensing refrigerant having a temperature lower than that of the control refrigerant to cause condensation of the gaseous product on the outer peripheral surface of the longitudinal pipe portion 4a of the condensing refrigerant pipe 4. In this manner, according to the above-described configuration, it is possible to achieve both the promotion of the direct FT reaction in the casing 2 and the condensation of the product.

Further, in the reactor 1, the longitudinal pipe portion cover 6 made of the punching plate is provided so as to cover the outer peripheral surface of the longitudinal pipe portion 4a passing through each catalyst holding portion 3c. Therefore, the liquid product attached to the outer peripheral surface of the longitudinal pipe portion 4a can fall along the outer peripheral surface of the longitudinal pipe portion 4a without leaking to the side provided with the catalyst 5, and can be recovered efficiently.

Further, the lower wall portion 13 of the casing 2 is provided with the post-reaction gas discharge port 13a at the center portion thereof, and is provided with the product taking-out port 13b at a predetermined position shifted in the radial direction from the center portion and different from that of the post-reaction gas discharge port 13a. Accordingly, the post-reaction gas remaining in the casing 2 can be smoothly discharged to the outside through the post-reaction gas discharge port 13a, and the liquid product can be smoothly taken out to the outside through the product taking-out port 13b without being mixed with the post-reaction gas.

Note that the present invention is not limited to the above-described embodiment, and can be carried out in various modes. For example, in the embodiment, the case where the direct FT reaction for producing hydrocarbon from the source gas containing H₂ and CO₂ is executed by the reactor 1 has been described, but the reactor of the present invention is not limited thereto, and can be applied to a reaction in which an exothermic reaction occurs in the casing 2 of the reactor 1, the source gas does not contain a liquid component at a reaction pressure at normal temperature, and the product contains a large amount of liquid component at a reaction pressure at normal temperature. For example, the reactor 1 can also be applied to a methanol synthesis reaction for producing CH₃OH (methanol) from a source gas containing H₂ and CO₂.

In the embodiment, the ten catalyst holding portions 3c of the control refrigerant pipe 3 and the ten longitudinal pipe portions 4a of the condensing refrigerant pipe 4 are provided in the reactor 1. However, the number of the catalyst holding portions 3c and the number of the longitudinal pipe portions 4a are not limited to particular ones, and may be one or a plurality of catalyst holding portions 3c and longitudinal pipe portions 4a other than ten depending on the size, shape, and the like of the reactor.

Furthermore, the detailed configurations and the like of the casing 2, the control refrigerant pipe 3, the condensing refrigerant pipe 4, the catalyst 5, and the longitudinal pipe portion cover 6 of the reactor 1 described in the embodiment are merely examples, and thus may be appropriately changed within the scope of the gist of the present invention.

### REFERENCE SIGNS LIST

1 Reactor
2 Casing
3 Control refrigerant pipe (first refrigerant flow path)
3a Inflow port
30 Outflow port
3c Catalyst holding portion
4 Condensing refrigerant pipe (second refrigerant flow path)
4a Longitudinal pipe portion (longitudinal flow path portion)
4b Lower lateral pipe portion
4c Upper lateral pipe portion
4d Inflow port
4e Outflow port
5 Catalyst
6 Longitudinal pipe portion cover (longitudinal flow path portion cover)
6a Upper end portion of longitudinal pipe portion cover
6b Top plate portion
6c Upper end cylindrical portion
6d Cover main body portion
7 Condensation space
11 Main body portion of casing
12 Upper wall portion of casing
12a Source gas introduction port
13 Lower wall portion of casing
13a Post-reaction gas discharge port
13b Product taking-out port
13c Product guiding path

1. A reactor for producing a predetermined product while a predetermined source gas is introduced and an exothermic reaction occurs due to a catalytic action of a predetermined catalyst, the reactor comprising:
a casing including a source gas introduction port into which the source gas is introduced and a product taking-out port for taking out the product produced, the casing being filled with the catalyst;
a first refrigerant flow path disposed in the casing so that a predetermined first refrigerant flows in the first refrigerant flow path to control a reaction temperature in the casing;
a second refrigerant flow path disposed in the casing so that a predetermined second refrigerant flows in the second refrigerant flow path to cause condensation of the product on an outer peripheral surface of the second refrigerant flow path; and
a product guiding path provided in the casing and guiding the product condensed and attached to the outer peripheral surface of the second refrigerant flow path to the product taking-out port,
wherein the casing includes a main body portion formed to have a tubular shape extending in an up-down direction, an upper wall portion which closes an upper end portion of the main body portion and is provided with the source gas introduction port. and a lower wall portion which closes a lower end portion of the main body portion and is provided with the product taking-out port,
wherein the second refrigerant flow path includes a plurality of longitudinal flow path portions extending in the up-down direction in the main body portion and through which the second refrigerant flows, a lower lateral flow path portion connecting lower end portions of the respective longitudinal flow path portions with each other and extending horizontally, and an upper lateral flow path portion connecting upper end portions of the respective longitudinal flow path portions with each other and extending horizontally,
wherein the first refrigerant flow path includes a plurality of catalyst holding portions each formed to surround each of the plurality of longitudinal flow path portions in a state of being spaced from an outer peripheral surface of each of the longitudinal flow path portions by a predetermined distance, and is configured such that the first refrigerant flows to an outer peripheral portion of each of the catalyst holding portions.
wherein the catalyst is filled between each of the longitudinal flow path portions and each of the catalyst holding portions,
wherein either the lower lateral flow path portion or the upper lateral flow path portion is provided with an inflow port into which the second refrigerant flows, and either the lower lateral flow path portion or the upper lateral flow path portion that is left is provided with an outflow port from which the second refrigerant flows out, and
wherein a positional relationship in the up-down direction is changeable between the inflow port and the outflow port.

2. The reactor according to claim 1, wherein a temperature of the second refrigerant is set to be lower than a temperature of the first refrigerant.

3. (canceled)

4. The reactor according to claim 1, wherein each of the catalyst holding portions is provided with a longitudinal flow path portion cover that has a tubular shape separating the catalyst in the catalyst holding portion from the longitudinal flow path portion in a state of extending along the longitudinal flow path portion and having a predetermined gap from the outer peripheral surface of the longitudinal flow path portion, and that allows passage of gas and prevents contact of the catalyst with the longitudinal flow path portion.

5. The reactor according to claim 4, wherein the longitudinal flow path portion cover is made of a punching plate in which a large number of through holes having a predetermined diameter are formed.

6. The reactor according to claim 1, wherein the casing further includes a post-reaction gas discharge port for discharging a post-reaction gas in the lower wall portion, and
wherein the product taking-out port is provided at a predetermined position shifted in a radial direction in the lower wall portion of the casing and different from that of the post-reaction gas discharge port.
